(19) United States
(12) Patent Application Publication
(10) Pub. No.: US 2018/0282360 A1
(43) Pub. Date: Oct. 4, 2018

(54) SWEETENER AND METHOD OF PRODUCTION THEREOF

(71) Applicant: Evonik Degussa GmbH, Essen (DE)

(72) Inventors: Olivier Zehnacker, Voecklabruck (AT); Thomas Tacke, Alzenau (DE); Thomas Haas, Muenster (DE); Nicole Brausch, Essen (DE); Marc Becker, Mobile (AL)

(73) Assignee: Evonik Degussa GmbH, Essen (DE)

(21) Appl. No.: 15/967,739

(22) Filed: May 1, 2018

Related U.S. Application Data
(63) Continuation of application No. 13/511,540, filed on May 23, 2012, now abandoned.

(30) Foreign Application Priority Data
Dec. 23, 2009 (DE) 10 2009 055 256.1

(51) Int. Cl. C07H 15/04; A23L 27/30

(52) U.S. Cl. CPC C07H 15/04; A23L 27/34; A23V 2250/6418; A23V 2250/642; A23V 2002/00

(57) ABSTRACT

A sweetener and to a method of production thereof is provided. The task of the present invention consists of providing a sucrose-based sweetener, in the production of which the step of separation of the residual sucrose from the isomerization stage is not required, and which has excellent properties for further processing, for example it can be formulated as sweets.

### FIELD OF THE INVENTION

The invention relates to a sweetener and to a method of production thereof.

### PRIOR ART

Isomalt (also called, isomaltitol, Palatinit®) is a sugar substitute, which is obtained from sucrose. It is produced in a two-stage process: first, sucrose is converted to isomaltulose (α-D-glucopyranosyl-1,6-fructose, also called Palatinose®) by rearrangement. The purified isomaltulose is then converted by catalytic hydrogenation to isomalt.

In the hydrogenation of isomaltulose, two isomers form: α-D-glucopyranosyl-1,1-D-mannitol (designated 1,1-GPM hereinafter) and α-D-glucopyranosyl-1,6-D-sorbitol (designated 1,6-GPS hereinafter), of which isomalt essentially consists.

The isomerization of sucrose to isomaltulose is as a rule carried out enzymatically with isomaltulose synthases (sucrose glucosylmutases, EC 5.4.99.11). DE1049800, DE2217628, EP 28900, EP49472 and EP 91063 describe methods with immobilized bacterial cells for enzymatic conversion of sucrose to isomaltulose. For this, EP 0625578 uses bacterial strains from the group comprising Protaminobacter rubrum (CBS 574.77), Serratia plymuthica (ATCC 15928), Serratia marcescens (NCIB 8285), Leuconostoc mesenteroides (NRRL-B 512 F (ATCC 1083 a)) and Erwinia rhapontici (NCPPB 1578). EP 0392556 and EP1257638 describe the use of bacterial strains from the group comprising Klebsiella terrigena JCM 1687, Klebsiella sp. No. 88 (FERM BP-2838) and Klebsiella singaporensis LX3 and LX21.

These isomerization processes are carried out with live or dead cells, with immobilized or free cells: thus, DE3133123 and EP0915986 describe for example methods of immobilization of the enzyme catalysts with calcium alginate or ion exchangers, and EP0001099 describes a method with free, live cells, which can produce isomaltulose in the course of fermentation.

Something that all the known methods of isomerization have in common is that the sucrose is never completely converted—traces can always be detected, and for further processing of the isomaltulose to isomalt, separation of the sucrose that has not been isomerized must be carried out.

For separation of the sucrose that has not been isomerized, as a rule crystallization of the isomaltulose is carried out. Methods of this kind are described for example in EP0091063 and EP1550666. EP0625578 describes a method in which removal of the sucrose that has not been isomerized is achieved by additional cleavage to the corresponding monosaccharides fructose and glucose and separation of them.

The hydrogenation of isomaltulose is common knowledge, and methods are described for example in GB1429334, DE2520173 and EP0625578, which use Raney nickel catalysts at elevated pressures and temperatures.

Moreover, methods are known from EP152779 and DE-A 4416115 for continuous hydrogenation of isomaltulose, which use unsupported moulded articles of elements of the 8th subgroup of the periodic system or unsupported moulded articles of elements of the iron subgroup of the 8th subgroup of the periodic system with elements of the 6th subgroup as catalysts.

EP0854148 describes a method of hydrogenation of isomaltulose on a catalyst containing nickel, nickel oxide and tungsten oxide.

EP0838468 describes a method of hydrogenation of isomaltulose on unsupported moulded articles containing alloys of elements of the iron subgroup of the VIIIth subgroup of the periodic system with elements of the IVth and/or Vth subgroup of the periodic system, serving as hydrogenation catalysts.

DE19523008 describes a method of hydrogenation of isomaltulose, for achieving defined ratios of 1,1-GPM to 1,6-GPS using a catalyst of ruthenium, nickel and mixtures thereof on an inert support.

DE19523008 describes a method of hydrogenation of isomaltulose on a catalyst containing ruthenium and/or nickel on an inert support for controlling the ratio of the isomers.

In the enzymatic conversion of sucrose, trehalulose (α-D-glucopyranosyl-1,1-fructose) and fructose and glucose are often formed as by-products, so depending on the purification carried out after the isomerization stage, these may enter the hydrogenation reaction. Trehalulose is converted there to α-D-glucopyranosyl-1,1-D-mannitol and to α-D-glucopyranosyl-1,1-D-sorbitol (designated 1,1-GPS hereinafter) and fructose and glucose to sorbitol and mannitol.

Sometimes, therefore, apart from the main constituents 1,1-GPM and 1,6-GPS, isomalt may also contain 1,1-GPS, mannitol and sorbitol.

Isomalt of this kind and methods of production thereof are described for example in JP-A 751079 and EP0625578.

One of the main disadvantages of all known methods of production of sucrose-based dietetic sweeteners such as isomalt is the need to separate the residual sucrose, which has strong glycaemic action, after the enzymatic isomerization of the starting sugar. EP0625578 describes this remaining residual sucrose aptly as explicitly “non-hydrogenable”.

In the separation of the residual sucrose by the methods described above, there are inevitably losses of isomaltulose or other valuable products.

The task of the present invention consists of providing a sucrose-based sweetener, in the production of which the step of separation of the residual sucrose from the isomerization stage is not required, and which has excellent properties for further processing, for example it can be formulated as sweets.

### DESCRIPTION OF THE INVENTION

It was found, surprisingly, that the sweetener described in Claim 1 and the method of production thereof described hereunder make a contribution to achieving the task stated above.

The present invention therefore relates to a sweetener based on sucrose as starting substance.

The invention further relates to a catalytic process, which permits the simultaneous hydrogenation of isomaltulose and optionally trehalulose to isomalt and sucrose to sorbitol and mannitol.

An advantage of the sweetener according to the invention is that in comparison with the conventional isomalt and relative to 1,1-GPM, it is enriched with 1,6-GPS, which has strong sweetening power and good dissolution in water; this is also the advantage of the method according to the invention, as it makes such a sweetener directly available as a product.

A further advantage of the method according to the invention is that it can be carried out at relatively low temperatures and pressures and therefore saves energy and resources.

The term “residual sucrose” means, in the context of the present invention, the sucrose fraction that was not converted in the reaction of the sucrose used initially with a sucrose mutase, and is present as sucrose alongside the sucrose isomerized to, for example, isomaltulose or trehalulose.

The term “sweetener” means, in the context of the present invention, a mixture of compounds, which can be in liquid or solid form, crystalline or dissolved, optionally can contain water and tastes sweet.

The term “acid support” means, in the context of the present invention, supports that are familiar to a person skilled in the art as an “acid support”, for example metal oxides, such as Al₂O₃, SiO₂, TeO₂ or mixed oxides thereof, which through its intrinsic properties displays acidity, but also said support that only has acid functionalities on the surface as a result of suitable treatment; they can for example be carrier materials that are treated with acids, e.g. phosphoric acid, or alternatively supports for which an acid functionality is only introduced on application of the active component ruthenium, e.g. as ruthenium chloride in acid solution; an acid support of this kind is for example an activated charcoal impregnated with ruthenium chloride in acid solution.

All percentages (%) stated are, unless stated otherwise, percentages by weight.

A contribution to solving the problems mentioned above is provided by a sweetener containing, preferably consisting of

20 wt. % to 75 wt. %, preferably 40 wt. % to 60 wt. %, especially preferably 45 wt. % to 57 wt. % of α-D-glucopyranosyl-1,6-D-sorbitol,
20 wt. % to 75 wt. %, preferably 40 wt. % to 60 wt. %, especially preferably 45 wt. % to 55 wt. % of α-D-glucopyranosyl-1,1-D-mannitol,
0.02 wt. % to 15 wt. %, preferably 0.1 wt. % to 10 wt. %, especially preferably 0.2 wt. % to 5 wt. % of α-D-glucopyranosyl-1,1-D-sorbitol,
0.02 wt. % to 15 wt. %, preferably 0.1 wt. % to 8 wt. %, especially preferably 0.2 wt. % to 3.5 wt. % of sorbitol and
0.02 wt. % to 15 wt. %, preferably 0.1 wt. % to 10 wt. %, especially preferably 0.2 wt. % to 2.9 wt. % of mannitol, in each case relative to the total amount of α-D-glucopyranosyl-1,1-D-mannitol, α-D-glucopyranosyl-1,6-D-sorbitol, α-D-glucopyranosyl-1,1-D-sorbitol, sorbitol and mannitol with the proviso, that the weight ratio of α-D-glucopyranosyl-1,6-D-sorbitol to α-D-glucopyranosyl-1,1-D-mannitol is greater than 1:1, preferably greater than 53:47, in particular greater than 55:45.

If the sweetener according to the invention consists of the aforementioned substances, the stated wt. % add up to 100.

For determination of the respective proportions by weight, it is possible to employ the methods described in the ISOMALT Specifications, elaborated within the scope of the 69th JECFA (2008), published in the FAO JECFA Monographs 5 (2008).

Preferably the sum of the wt. % of α-D-glucopyranosyl-1,6-D-sorbitol and α-D-glucopyranosyl-1,1-D-mannitol is greater than 75, preferably greater than 80, especially preferably greater than 86 relative to the total weight of dry matter of the sweetener.

Preferably the sweetener according to the invention contains less than 2.5 wt. %, in particular less than 0.3 wt. %, and most preferably no detectable amounts of sucrose, relative to the total weight of dry matter of the sweetener.

A further contribution to solving the problems mentioned above is provided by a method of production of a sweetener by reaction of a carbohydrate mixture containing isomaltulose, sucrose and optionally trehalulose, fructose and glucose and/or other polysaccharides with hydrogen, characterized in that the reaction is carried out in the presence of at least one catalyst, which is based on ruthenium (Ru) and/or at least one oxide of ruthenium.

In this method both the isomaltulose and optionally trehalulose are preferably hydrogenated catalytically by hydrogen to 1,1-GPM and 1,6-GPS and optionally to 1,1-GPS and sucrose is cleaved to fructose and glucose and these are hydrogenated to mannitol and sorbitol. The two last-mentioned are also sugar substitutes and are therefore ideal coproducts with the 1,1-GPM, 1,6-GPS and 1,1-GPS obtained.

Therefore the reaction in the method according to the invention corresponds to a catalytic hydrogenation accompanied by cleavage of the sucrose to fructose and glucose.

It is therefore preferable for the cleavage of the sucrose and the hydrogenation of the other carbohydrates present to take place simultaneously.

In the methods according to the invention, preferably catalysts are used in which ruthenium (Ru) and/or the ruthenium-containing compound are immobilized on a support, in particular an acid support or carbon-containing support.

Reaction preferably takes place in an aqueous solution, so that the carbohydrate mixture can contain water. Preferably the carbohydrate mixture therefore contains 20 wt. % to 80 wt. %, preferably 30 wt. % to 70 wt. %, especially preferably 40 wt. % to 60 wt. % of water relative to the total carbohydrate mixture.

The pH of the aqueous solution is preferably in the neutral or acid range, corresponding to a pH below 8.

The carbohydrate mixture used in the method according to the invention is preferably obtainable by the enzymatic reaction of sucrose-containing, aqueous solutions, for example aqueous solutions of sugar from sugar beet or sugar cane, with isomaltulose synthases. Suitable isomaltulose synthases are for example those from Enterobacter sp. strain FMB1, Erwinia rhapontici, Klebsiella planticola strain UQ14S, Klebsiella pneumoniae NK33-98-8, Klebsiella sp. LX3, Pantoea dispersa UQ68J, Protaminobacter ruber Z12, Protaminobacter rubrum, Pseudomonas mesoacidophila MX-45, Serratia plymuthica. In particular, carbohydrate mixtures obtainable by the enzymatic reaction of sucrose-containing, aqueous solutions with isomaltulose synthases from Protaminobacter rubrum, in particular of the strain Protaminobacter rubrum CBS 574.77, can be used advantageously in the method according to the invention.

The sucrose contained in the carbohydrate mixture is therefore preferably residual sucrose.

The carbohydrate mixture used in the method according to the invention preferably contains 0.01 wt. % to 15 wt. %, preferably 0.1 wt. % to 5 wt. % and especially preferably 0.2 wt. % to 2 wt. % of sucrose relative to the dry weight of the total carbohydrate mixture.

The carbohydrate mixture used in the method according to the invention preferably contains at least 70 wt. %, preferably at least 80 wt. % and most preferably at least 90 wt. % of isomaltulose relative to the dry weight of the total carbohydrate mixture.

The carbohydrate mixture used in the method according to the invention preferably contains 0.02 wt. % to 30 wt. %, preferably 0.1 wt. % to 20 wt. %, especially preferably 0.2 wt. % to 10 wt. % of trehalulose relative to the dry weight of the total carbohydrate mixture.

The aforementioned catalysts based on ruthenium (Ru) and/or ruthenium oxide have, surprisingly, proved to be far superior to other known hydrogenation catalysts with respect to complete conversion of the educts used and to extremely high selectivity for the aforementioned products.

All solids that appear to a person skilled in the art to be suitable can be considered as catalyst supports. These are for example carbon, for example in the form of activated charcoal, and in particular also acid supports, for example metal oxides, such as Al₂O₃, SiO₂, TeO₂, mixed oxides thereof or also MgO—SiO₂, ZrO₂—SiO₂ and heteropolyacids. We may also mention: mineral acids, for example H₃PO₄ or H₂SO₄, which are applied to solid, preferably porous, also preferably inert supports, cation exchangers, salts of oxygen-containing mineral acids, preferably of heavy metals (phosphates, sulphates, tungstates), halides of trivalent metals (such as AlCl₃) on porous supports, zeolites (H form) or the so-called, H₂SO₄-treated super acids ZrO₂ or TiO₂.

Supports that are rather to be classified as neutral on the basis of their functionality are also suitable, for example activated charcoal or TiO₂, which preferably acquire acid functionality by a suitable impregnation process and/or by application of the catalyst metal itself.

In this connection it is preferable if these supports have suitable pore volumes, which are suitable for good binding and uptake of the hydrogenation catalyst. Moreover, total pore volumes according to DIN 66133 in a range from 0.01 to 3 ml/g are preferred, and those in a range from 0.2 to 1 ml/g are especially preferred. Moreover, it is preferable if the solids suitable as supports have a surface area in a range from 0.001 to 1500 m²/g, preferably in a range from 10 to 450 m²/g and more preferably in a range from 10 to 270 m²/g in the BET test according to DIN 66131. On the one hand, a loose product that has an average particle diameter in a range from 0.1 to 40 mm, preferably in a range from 0.8 to 7 mm and more preferably in a range from 1.5 to 7 mm can be used as support for the hydrogenation catalyst. Furthermore, the wall of the hydrogenation reactor can serve as inert support.

Dipping or impregnation or incorporation in a carrier matrix may be mentioned in particular as techniques for applying the hydrogenation catalyst.

In one embodiment of the invention, the acid support consists at least partially of an oxide compound. These oxide compounds should have at least one of the elements selected from the group comprising Si, Ti, Te, Zr, Al, P or a combination of at least two of these elements.

Preferred acid supports are selected from the group comprising, and preferably consisting of, silicon, aluminium, tellurium and phosphorus oxides, with Al₂O₃, SiO₂, TeO₂ and mixed oxides thereof being especially preferred and Al₂O₃ being quite especially preferred.

Super-acid supports can also be used as supports in the method according to the invention.

These supports are known as such by a person skilled in the art, for example zeolites of the H—Y type, preferably with an Si—Al ratio >50, and acid ion exchangers with appropriate temperature resistance, such as those available under the trade name Amberlyst.

In an alternative embodiment of the method according to the invention, neutral supports can also be used as supports. These are in particular selected from the list comprising elemental carbon, in particular activated charcoal, and TiO₂, with activated charcoal being especially preferred.

The method according to the invention is advantageously carried out at elevated temperatures. The preferred temperature range is 80° C. to 150° C., the process temperature being regarded as the temperature measured in the carbohydrate mixture, which optionally already contains the sweetener according to the invention.

An alternative embodiment of the method according to the invention is characterized in that the process is carried out up to a conversion of 50% to 95% relative to the hydrogenation of the isomaltulose in a temperature range between 80 to 120° C. and the further, essentially 100% conversion relative to the hydrogenation of the isomaltulose in a temperature range between 100° C. to 150° C., preferably 121° C. to 150° C.

In this connection, it is preferable according to the invention for the two different temperature ranges to be spatially separate from one another, using in both temperature ranges a catalyst in which ruthenium (Ru) and/or the ruthenium-containing compound is immobilized on an oxide-containing support, the oxide being selected in particular from Al₂O₃ and TiO₂.

In an alternative embodiment, in this connection it is preferable according to the invention for the two different temperature ranges to be spatially separate from one another, in the temperature range from 80° C. to 120° C., using a catalyst in which ruthenium (Ru) and/or the ruthenium-containing compound is immobilized on an oxide-containing support, the oxide being selected in particular from Al₂O₃ and TiO₂, and in the temperature range from 100° C. to 150° C., preferably 121° C. to 150° C., using a catalyst in which ruthenium (Ru) and/or the ruthenium-containing compound is immobilized on a carbon-containing support.

A special embodiment of the method according to the invention is characterized in that a super-acid support is used as the support and the process temperature is below 120° C., in particular 80° C. to 110° C.

Regarding the pressure during the hydrogenation reaction, in particular a pressure of at least 15 bar, preferably of at least 30 bar, especially preferably at least 40 bar, has proved to be advantageous. Values between 40 bar and 150 bar, in particular between 40 bar and 90 bar, for example in the range from about 50 to 60 bar, are especially preferable.

Preferably the method is carried out until sucrose can no longer be detected in the sweetener obtained.

For the sweetener obtained by the method according to the invention, which is preferably as a carbohydrate mixture in liquid form, to be converted to the dry form, the water present as solvent can be removed using an evaporator or a dryer, for example a down-flow evaporator or a drum dryer or a spray dryer.

It may be advantageous for the sweetener obtained to be further processed with additional purification or enrichment and/or depletion steps.

Thus, it may be advantageous to lower the content of mannitol by applying another crystallization step, for example to 0.02-15 wt. %, preferably to 0.1-10 wt. %, especially preferably to 0.2-2.9 wt. % relative to the dry weight of the sweetener; this is easily possible owing to the low water solubility of mannitol.

In the examples given below, the present invention is described for purposes of illustration, but the invention, the scope of which follows from the full description and the claims, is not limited to the embodiments presented in the examples.

### EXAMPLES

### Example 1: Ru-Catalysed Hydrogenation of an Isomaltulose Solution Containing Sucrose at 90° C.

An aqueous solution containing 40 wt. % isomaltulose and 3 wt. % sucrose is hydrogenated according to the invention on an Ru-catalyst, 1.5 wt. % on aluminium oxide, at 60 bar hydrogen and 90° C. in a continuously operated fixed-bed reactor at an LHSV (liquid hourly space velocity) of 0.47 h⁻¹. The apparatus consisted of a tubular reactor with air heating or air cooling with an inside diameter of the reactor tube of 11 mm. The tube was packed with 19 ml of catalyst Noblyst® 3001, Evonik Degussa GmbH.

The hydrogen volume flow rate was 100 Nml/min.

The product shows an isomer ratio 1,6-GPS to 1,1-GPM of 56:44 at a conversion of 80% with respect to the isomaltulose used and 24% with respect to sucrose. The reacted isomaltulose was hydrogenated to isomalt at almost 100% selectivity. In the reaction conditions, the sucrose is hydrogenated to mannitol and sorbitol.

### Example 2: Ru-Catalysed Hydrogenation of an Isomaltulose Solution Containing Sucrose at 120° C.

If the same solution as used in example 1 is reacted in this case at 120° C., in otherwise identical conditions, the isomaltulose used is hydrogenated to 95%, and the sucrose to 93%. Once again, the reacted isomaltulose was hydrogenated to isomalt at almost 100% selectivity. Furthermore, the isomer ratio 1,6-GPS to 1,1-GPM reached 56:44.

In the reaction conditions, the sucrose is hydrogenated to mannitol and sorbitol.

### Example 3: Ru-Catalysed (Ru/C) Hydrogenation of an Isomaltulose Solution Containing Sucrose at 90° C.

An aqueous solution containing 40 wt. % isomaltulose and 3 wt. % sucrose is hydrogenated according to the invention on an Ru-catalyst, 2 wt. % on activated charcoal, at 60 bar hydrogen and 90° C. in a continuously operated fixed-bed reactor at an LHSV of 0.47 h⁻¹. The apparatus consisted of a tubular reactor with air cooling with an inside diameter of the reactor tube of 11 mm. The tube was packed with 19 ml of catalyst Noblyst® 3000, Evonik Degussa GmbH.

The hydrogen volume flow rate was 100 Nml/min.

The product shows an isomer ratio 1,6-GPS to 1,1-GPM of 56:44 at a conversion of 96% with respect to the isomaltulose used and 60% with respect to sucrose. The reacted isomaltulose was hydrogenated to isomalt at almost 100% selectivity. In the reaction conditions, the sucrose is hydrogenated to mannitol and sorbitol.

### Example 4: Ru-Catalysed (Ru/C) Hydrogenation of An Isomaltulose Solution Containing Sucrose at 120° C.

If the same solution as used in example 3 is reacted at 120° C., but in otherwise identical conditions, the isomaltulose used is hydrogenated to >99%, and the sucrose to 98%. Once again, the reacted isomaltulose was hydrogenated to isomalt at almost 100% selectivity. Furthermore, the isomer ratio 1,6-GPS to 1,1-GPM reached 56:44.

In the reaction conditions, the sucrose is hydrogenated to mannitol and sorbitol.

### Example 5: Ni-Catalysed Hydrogenation of an Isomaltulose Solution Containing Sucrose at 90° C., Not According to the Invention

An aqueous solution containing 40 wt. % isomaltulose and 3 wt. % sucrose is hydrogenated with 10.5 g of a Raney-Ni catalyst, B 113 W, Evonik Degussa GmbH, at 60 bar hydrogen and 90° C. in a stirred tank reactor. The apparatus consisted of a Parr RK2 stirred tank reactor with gassing stirrer, a nominal volume of 1.8 L and a reaction volume of 1.2 L; hydrogenation took place isothermally without basket in the slurry.

After 4 h there is complete conversion of the isomaltulose to 1,6-GPS and 1,1-GPM at an isomer ratio of 53:47, but conversion of the sucrose is not detected in this period of time. The reacted isomaltulose was hydrogenated to isomalt at almost 100% selectivity.

1. A sweetener comprising
20 wt. % to 75 wt. %, of α-D-glucopyranosyl-1,6-D-sorbitol,
20 wt. % to 75 wt. %, of α-D-glucopyranosyl-1,1-D-mannitol,
0.02 wt. % to 15 wt. %, of α-D-glucopyranosyl-1,1-D-sorbitol,
0.02 wt. % to 15 wt. %, and
0.02 wt. % to 15 wt. %, of mannitol,
in each case relative to the total amount of α-D-glucopyranosyl-1,1-D-mannitol, α-D-glucopyranosyl-1,6-D-sorbitol, α-D-glucopyranosyl-1,1-D-sorbitol, sorbitol and mannitol
with the proviso,
that the weight ratio of α-D-glucopyranosyl-1,6-D-sorbitol to α-D-glucopyranosyl-1,1-D-mannitol is greater than 1:1.

2. A method of production of a sweetener, comprising reacting a carbohydrate mixture containing isomaltulose and sucrose, in the presence of at least one catalyst, which is based on ruthenium (Ru) and/or at least one oxide of ruthenium.

3. The method according to claim 2, wherein the carbohydrate mixture contains 0.01 wt. % to 15 wt. % of sucrose relative to the dry weight of the total carbohydrate mixture.

4. The method according to claim 2, wherein the carbohydrate mixture contains 0.02 wt. % to 30 wt. % of trehalulose relative to the dry weight of the total carbohydrate mixture.

5. The method according to claim 2, wherein the carbohydrate mixture contains 20 wt. % to 70 wt. % of water relative to the total carbohydrate mixture.

6. The method according to claim 2, wherein in the catalyst, the ruthenium (Ru) and/or the ruthenium-containing compound is immobilized on a support.

7. The method according to claim 6, wherein the total pore volume of the support according to DIN 66133 is in a range from 0.01 to 3 ml/g.

8. The method according to claim 6, wherein the support has a surface area in a range from 0.001 to 1500 m²/g in the BET test according to DIN 66131.

9. The method according to claim 6, wherein the support is a neutral support, in particular TiO₂ or activated charcoal.

10. The method according to claim 6, wherein the support is selected from the group comprising acid oxides and mixed oxides, natural and synthetic silicates.

11. The method according to claim 6, wherein the support consists at least partially of an oxide compound of at least one of the elements selected from the group comprising Si, Ti, Te, Zr, Al, P or a combination of at least two of these elements, in particular of Al₂O₃.

12. The method according to claim 6, wherein the support is a super-acid support selected from zeolites of the H—Y type or from acid ion exchangers.

13. The method according to claim 2, wherein the method is carried out in a temperature range from 80° C. to 150° C.

14. The method according to claim 12, which is carried out at a temperature below 120° C.

15. The method according to claim 2, which is carried out up to a conversion of 50% to 95% relative to the hydrogenation of the isomaltulose in a temperature range between 80 to 120° C. and the further, essentially 100% conversion relative to the hydrogenation of the isomaltulose in a temperature range between 100° C. to 150° C.

16. The method according to claim 15, wherein the two different temperature ranges are spatially separate from one another, and in both temperature ranges a catalyst is used in which ruthenium (Ru) and/or the ruthenium-containing compound is immobilized on an oxide-containing support, the oxide being selected in particular from Al₂O₃ and TiO₂.

17. The method according to claim 15, wherein the two different temperature ranges are spatially separate from one another, and in the temperature range from 80° C. to 150° C., a catalyst is used in which ruthenium (Ru) and/or the ruthenium-containing compound is immobilized on an oxide-containing support, the oxide being selected in particular from Al₂O₃ and TiO₂, and in the temperature range from 100° C. to 150° C., a catalyst is used in which ruthenium (Ru) and/or the ruthenium-containing compound is immobilized on a carbon-containing support.

18. The method according to claim 2, wherein the pressure used during the process is at least 15 bar.
